(19) United States
(12) Patent Application Publication
(10) Pub. No.: US 2007/0049776 A1
(43) Pub. Date: Mar. 1, 2007

(54) Preparation of Monofluoromethyl Ether

(75) Inventors: Zhiquan Zhao, Linyi city (CN); Lizeng Peng, Linyi City (CN); Wenli Ti, Linyi City (CN)

Correspondence Address: NORRIS, MCLAUGHLIN & MARCUS, P.A., 875 THIRD AVE, 18TH FLOOR, NEW YORK, NY, 10022, US

(73) Assignee: SHANDONG NEWTIME PHARMACEUTICALS CO., LTD, 273400 (CN)

(21) Appl. No.: 11/467,243

(22) Filed: Aug. 25, 2006

(30) Foreign Application Priority Data
Aug. 26, 2005 (CN) 200510093352.7

(51) Int. Cl. C07C 43/17

(52) U.S. Cl. 568/683

(57) ABSTRACT

The invention relates to a method for preparing a monofluoromethyl ether, in particular fluoromethyl 2,2,2-trifluoro-1-(trifluoromethyl)ethyl ether (i.e. Sevoflurane), wherein hexafluoroisopropanol is used as a reactant reacting with trioxymethylene in the presence of fluorine-containing catalyst.

### FIELD OF THE INVENTION

This invention relates to a method for preparing a monofluoromethyl ether, in particular fluoromethyl 2,2,2-trifluoro-1-(trifluoromethyl)ethyl ether (i.e. Sevoflurane).

### BACKGROUND ART

In recent years, several fluorinated ethers have been found to be useful as inhalation anesthetics, notably desflurane (CF₃CHFOCHF₂), isoflurane (CF₃CHClOCHF₂), enflurane (ClFCHCF₂OCHF₂), and sevoflurane ((CF₃)₂CHOCH₂F). Sevoflurane is an advantageous inhalation anesthetic because of its rapid loss of consciousness and rapid recovery, which are desirable characteristics of modern inhalation anesthetics. Sevoflurane is administered by inhalation route to warm blooded, air-breathing animals in amount of about 1-5 vol. % in admixture with oxygen or a gaseous mixture containing oxygen in amount sufficient to support respiration.

U.S. Pat. Nos. 3,683,092 and 3,689,571 disclosed the use of sevoflurane as an inhalation anesthetic and its preparation by the reaction of chloromethyl 2,2,2-trifluoro-1-(trifluoromethyl)ethyl ether with excess of potassium fluoride in a solvent with high boiling point at 120° C. to replace the chlorine of the chloromethyl group with fluorine. These patents also disclosed a method for preparing sevoflurane by the reaction of hexafluoroisopropanol with dimethyl sulfate in sodium hydroxide solution, and subsequent fluorination of the resulting methyl 2,2,2-trifluoro-1-(trifluoromethyl)ethyl ether with bromine trifluoride. U.S. Pat. No. 4,328,376 disclosed a method for isolating sevoflurane from a by-product olefin produced in a process similar to that described in U.S. Pat. No. 3,689,571.

Other synthetic routes for preparing sevoflurane are found in the following patent publications: U.S. Pat. No. 3,897,502 (fluorination of methyl 2,2,2-trifluoro-1-(trifluoromethyl)ethyl ether with 20% fluorine in argon); U.S. Pat. Nos. 4,250,334 and 4,469,898 (fluoromethylation of hexafluoroisopropanol by use of hydrogen fluoride, formaldehyde and sulfuric acid or other dehydrating agents); and PCT Application WO 97/25,303 (reaction of hexafluoroisopropanol with bis(fluoromethyl)ether).

Okazaki, et al. (J. Fluorine Chem. 1974, 4(4), 387) described an electrochemical fluorination for obtaining fluoromethyl ethers. German Patent DE25 20 962 provides a synthesis of fluoromethyl ethers from chloromethyl ethers and hydrogen fluoride at 125-149° C. in the presence of chromium oxyfluoride. Bensoam, et al. (Tetrahedron Lett. 1979, 4, 353) described a synthesis of fluoromethyl ethers by halogen exchange with tetrahydroxyfluorophosphoranes. German Patent DE28 23 969 disclosed a process for preparing organofluorine compounds including monofluoromethyl ethers, by reaction of corresponding organochlorides or organobromides with selected amine hydrofluorides. Triethylamine hydrofluoride and piperidine hydrofluoride are specific examples of fluorinating agents used for the preparation of such organofluorine compounds, which are typically produced in yields of about 40-80%. Chinese Patent CN1744187 provided an improvement of the process disclosed by DE28 23 969, and achieves good effect.

Additionally, U.S. Pat. No. 4,874,901 described a reaction of chloromethyl 2,2,2-trifluoro-1-(trifluoromethyl)ethyl ether and pure potassium fluoride, but the production yield is undesirable.

In accordance with the present invention, a method for preparing certain monofluoromethyl ethers, in particular sevoflurane, is provided, which is characterized by improved yields over the methods in prior arts.

### SUMMARY OF THE INVENTION

The present invention relates to a method for catalyzing the reaction of an alcohol and trioxymethylene in the presence of a catalyst to yield the corresponding monofluoromethyl ether. More particularly, the invention relates to a method for preparing fluoromethyl 2,2,2-trifluoro-1-(trifluoromethyl)ethyl ether (i.e. sevoflurane), with the reaction of hexafluoroisopropanol (HFIP, (CF₃)₂CH—OH) and trioxymethylene in the presence of a catalyst. The present invention generally relates to a method for catalyzing the reaction of an alcohol and trioxymethylene in the presence of fluorine-containing catalyst to yield the corresponding monofluoromethyl ether. The alcohol starting materials of the present invention is a known compound and is represented by the following general formula: 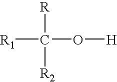
wherein R is fluoro lower alkyl, fluorine or chlorine, R₁ and R₂ are independently selected from hydrogen, lower alkyl, branched lower alkyl, fluoro lower alkyl, fluorine and chlorine, with the proviso that at least one of R, R₁ or R₂ is fluoro lower alkyl, lower alkyl or branched lower alkyl. One of the above alcohols and trioxymethylene is reacted together in the presence of a catalyst to produce monofluoromethyl ether represented by formula I: 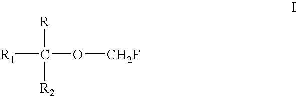
wherein R, R₁ or R₂ is as defined above.

In a preferred embodiment, the present invention relates to a method for preparing fluoromethyl 2,2,2-trifluoro-1-(trifluoromethyl)ethyl ether (i.e. sevoflurane) represented by the following formula by catalyzing the reaction of hexafluoroisopropanol (HFIP) and trioxymethylene in the presence of the catalyst:

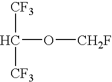

A useful catalyst of the present invention can be selected from MF₃ and the complex thereof, wherein M is boron (B) or aluminium (Al). A typical catalyst according to the invention can be BF₃ (boron trifluoride), AlF₃ (aluminium trifluoride), or BF₃.Et₂O (boron trifluoride-ether complex).

A useful catalyst of the present invention is used in amount of 1-15 mol % of an alcohol. In a preferred embodiment, the catalyst is used in amount of 8 mol % of the alcohol.

The term “lower alkyl” used herein means a saturated alkyl group containing 1-6 carbons and is linear unless specifically stated otherwise. The term “fluoro lower alkyl” used herein refers to a saturated alkyl group containing 1-6 carbons substituted with at least one fluorine atom. Preferred fluoro lower alkyl group is trifluoromethyl.

A useful catalyst in the reaction of the present invention is known in the art of chemistry. It is useful to provide high yield of the reaction by use of the catalyst mentioned herein. The preferred catalyst of the invention is BF₃.Et₂O. Additionally, in accordance with the present invention, the catalyst is used with certain molar amount sufficient to catalyze the reaction of the mixture of an alcohol and trioxymethylene, and the reactant mixture is heated for a period of time sufficient to cause the formation of the product monofluoromethyl ether.

The conversion reaction of an alcohol and trioxymethylene to a monofluoromethyl ether by catalyzing can be conducted in the absence of a solvent, or in the presence of a solvent, for example, a low boiling point chlorine-containing solvent, such as dichloromethane, chloroform, 1,2-dichloroethane, or the like. In a preferred embodiment of the invention, the reaction is conducted in the presence of or absent of a solvent with suitable or excessive alcohol which advantageously also functions as a solvent. The conversion of an alcohol to a monofluoromethyl ether in accordance with the present invention can be conducted at elevated temperatures in the range of 25-100° C., at atmospheric pressure or in a sealed pressure vessel. In a preferred embodiment of the invention, the reaction is conducted at reflux temperature of an alcohol.

The use of a catalyst for the conversion of an alcohol to monofluoromethyl ether in the present invention shows a substantial improvement comparing with the previous methods without any use of a catalyst. The production yield or the conversion yield of the desired product in the present invention is consistently higher than that obtained without use of a catalyst, which is shown in the following Examples. The method of the invention with high yield has heretofore not been described in any literature documents.

### DETAILED DESCRIPTION OF PREFERRED EMBODIMENTS OF THE INVENTION

The following non-limiting examples illustrate the method of the invention.

### EXAMPLE 1

HFIP (168 g), BF₃.Et₂O (11.3 g) and trioxymethylene (90 g) were added into a one-necked flask (250 mL) equipped with a magnetic stirrer and a reflux unit, then the mixture is heated to reflux for 5 hrs till the completion of the reaction. After that, the reaction mixture is fractionated, and the fraction is collected at 58.5-58.6° C. (the boiling point of sevoflurane) in amount of 186 g. The fraction contains 90.5% of sevoflurane by GC analysis. The yield of producing sevoflurane is 84%.

### EXAMPLE 2

HFIP (168 g), AlF₃ (anhydrous, 6.7 g), chloroform (120 mL) and trioxymethylene (90 g) were added into a one-necked flask (500 mL) equipped with a magnetic stirrer and a reflux unit, then the mixture is heated to reflux for 5 hrs till the completion of the reaction. After that, the reaction mixture is fractionated, and the fraction is collected at 58.5-58.6° C. (the boiling point of sevoflurane) in amount of 164 g. The fraction contains 88.2% of sevoflurane by GC analysis. The yield of producing sevoflurane is 72%.

Various modifications or adaptations to the foregoing examples without departing from the spirit and scope of the invention are apparent for one skilled in the art, which are also included in the scope of the invention.

1. A method for preparing monofluoromethyl ether represented by formula I by catalyzing the reaction of an alcohol and trioxymethylene in the presence of a catalyst: 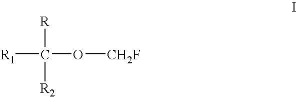
wherein
R is fluoro lower alkyl, fluorine or chlorine, R₁ and R₂ are independently selected from hydrogen, lower alkyl, branched lower alkyl, fluoro lower alkyl, fluorine and chlorine, with the proviso that at least one of R, R₁ and R₂ is fluoro lower alkyl, lower alkyl or branched lower alkyl.

2. A method according to claim 1, wherein the monofluoromethyl is sevoflurane.

3. A method according to claim 1, wherein the catalyst is MF₃ or the complex thereof, and M is Al or B.

4. A method according to claim 3, wherein the catalyst is BF₃.Et₂O.

5. A method according to claim 1, wherein the catalyst is in amount of 1-15 mol % of the alcohol.

6. A method according to claim 5, wherein the catalyst is in amount of 8 mol % of the alcohol.

7. A method according to claim 1, wherein the reaction is conducted in the presence of a solvent.

8. A method according to claim 7, wherein the reaction is conducted at reflux temperature of the reaction mixture.

9. A method according to claim 1, wherein the reaction is conducted in the absence of a solvent but with excess of the alcohol.

10. A method according to claim 9, wherein the reaction is conducted at reflux temperature of the reaction mixture.

11. A method according to claim 2, wherein the alcohol is hexafluoroisopropanol, and the catalyst is BF₃.Et₂O; the reaction is conducted in the absence of a solvent but with excess of the alcohol at the reflux temperature; after the completion of the reaction, the product sevoflurane is collected by fractionation.

12. A method according to claim 2, wherein the alcohol is hexafluoroisopropanol, and the catalyst is anhydrous AlF₃; the reaction is conducted in the presence of a solvent at the reflux temperature; after the completion of the reaction, the product sevoflurane is collected by fractionation.

13. A method according to claim 12, wherein the solvent is chloroform.
